(19) United States
(12) Patent Application Publication
(10) Pub. No.: US 2014/0275294 A1
(43) Pub. Date: Sep. 18, 2014

(54) DEVICES AND METHODS FOR BIOMARKER DETECTION PROCESS AND ASSAY OF LIVER INJURY

(71) Applicant: BANYAN BIOMARKERS, INC., Alachua, FL (US)

(72) Inventors: Stanislav I. Svetlov, Alachua, FL (US); Ronald L. Hayes, Alachua, FL (US)

(73) Assignee: BANYAN BIOMARKERS, INC., Alachua, FL (US)

(21) Appl. No.: 14/217,200

(22) Filed: Mar. 17, 2014

Related U.S. Application Data
(60) Provisional application No. 61/798,146, filed on Mar. 15, 2013.

(51) Int. Cl. G01N 33/543

(52) U.S. Cl. CPC G01N 33/54306
USPC 514/789; 422/69; 435/287.2; 436/501; 435/7.92; 435/7.94

(57) ABSTRACT

An in vitro diagnostic (IVD) device is used to detect the presence of and/or severity of liver injury in a subject. The IVD device relies on an immunoassay which identifies biomarkers that are diagnostic of liver injury in a biological sample, such as whole blood, plasma, serum. The inventive IVD device may measure one or more of several specific markers in a biological sample and output the results to a machine readable format wither to a display device or to a storage device internal or external to the IVD.

### CROSS-REFERENCE TO RELATED APPLICATIONS

The present application claims priority of U.S. Provisional patent application No. 61/798,146 filed on Mar. 15, 2013 the content of which is herein incorporated by reference.

### FIELD OF THE INVENTION

The invention provides for an in vitro diagnostic device and software which enables the reliable detection of damage to the liver of an individual through biomarker identification. These devices and software methods provide simple yet sensitive clinical approaches to diagnosing liver damage using biological fluid particularly measuring for one or more of a biomarker such as Argininosuccinate synthetase (ASS), argininosuccinate lyase (ASL), sulfuration (estrogen sulfotransferase (EST), squalene synthase (SQS), liver glycogen phosphorylase (GP), carbamoyl-phosphate synthetase (CPS-1), α-enolase 1, SULT2A1, glucose-regulated protein (GRP). Inventive markers include proteins; or protein fragments; autoantibodies; DNA; RNA; or miRNA.

### BACKGROUND OF THE INVENTION

The liver is an extremely important organ. As the major metabolic organ of the body, the liver plays some role in almost every biochemical process, including the deamination of amino acids and the formation of urea, the regulation of blood sugar through the formation of glycogen, the production of plasma proteins, the production and secretion of bile, phagocytosis of particulate matter from the splachnic (intestinal) circulation, and the detoxification and elimination of both endogenous and exogenous toxins.

The many functions of the liver depend on its intimate association with circulating blood. Each liver cell is exposed on at least one face to a blood sinusoid which contains oxygenated arterial blood mixed with venous blood from the splanchnic circulation. This profuse blood supply is necessary for the liver to function. The blood from the sinusoids supplies the hepatocytes with oxygen and nutrients. The hepatocytes use the nutrients both for their own metabolic needs and for the synthesis of the liver's many essential products. Abnormalities in the blood or vasculature can have immediate and severe effects on the liver. For example, liver cells are exposed to high concentrations of any toxic compounds that are ingested orally, such as ethyl alcohol. Even when the ingested compound is not itself toxic, intermediate derivatives produced during hepatic metabolism of the compound may damage the hepatocytes. This phenomenon occurs, for example, in carbon tetrachloride poisoning. Since the blood moves slowly through hepatic sinusoids, liver cells are also quite vulnerable to blood-borne infectious agents such as viruses and bacteria. Furthermore, derangements in hepatic blood pressure can damage liver tissue. Right-sided cardiac failure increases hepatic blood pressure and can lead to pressure necrosis (hepatocellular death) and fibrosis. Left-sided cardiac failure can reduce hepatic perfusion and lead to hepatocellular anoxia and death.

Liver damage from any source may result in liver regeneration, necrosis (cell death), degeneration, inflammation, fibrosis, or mixtures of these processes, depending on the type and extent of injury and its location within the liver. The liver has great functional reserves, but with progressive injury, disruption of liver function can have life-threatening consequences. Cirrhosis, which is a type of end-stage liver disease, is one of the top ten causes of death in the Western world.

Biomarkers of liver damage and sepsis have been identified. Argininosuccinate synthetase (ASS), argininosuccinate lyase (ASL), sulfuration (estrogen sulfotransferase (EST), squalene synthase (SQS), liver glycogen phosphorylase (GP), carbamoyl-phosphate synthetase (CPS-I), α-enolase 1, glucose-regulated protein (GRP) and spectrin breakdown products, all have been identified as protein biomarkers correlating the detection of liver injury. In addition, there are a few markers of liver injury (e.g. ALT, LDH), which have been used for diagnostics or monitoring of clinical conditions where liver injury, such as, ischemia/reperfusion is a major pathogenic cause of liver damage. The nature of these biomarkers is detailed in U.S. Pat. No. 7,645,584, US 2010/0196942 A1 and U.S. Pat. No. 8,048,638, the contents of which are hereby incorporated by reference. However, there remains to be any type of method of using these biomarkers in the clinical environment.

Thus, there exists a need for a process and an assay for providing improved measurement of liver damage through the quantification of biomarkers in the clinical environment, whether alone or in combination with another biomarker associated with the specific condition.

There is also an unmet need for clinical intervention through the use of an in vitro diagnostic device to identify these biomarkers of liver damage so that subject results may be obtained rapidly in any medical setting to direct the proper course of treatment for subjects suffering from a liver injury.

### SUMMARY OF THE INVENTION

The present invention provides an in vitro diagnostic device specifically designed and calibrated to detect protein markers that are present in the samples of patients suffering from liver damage. These devices present a sensitive, quick, and non-invasive method to aid in diagnosis of liver injuries by detecting and determining the amount of biomarkers that are indicative to the respective injury. The measurement of these markers, alone or in combination of other markers for the injury type, in patient samples provides information that a diagnostician can correlate with a probable diagnosis of the extent of an injury such as liver cirrhosis, hepatitis and sepsis.

In certain inventive embodiments, an in vitro diagnostic device is provided to measure biomarkers that are indicative of liver injury, liver failure, liver transplant damage, liver disease, liver damage due to drug or alcohol addiction or exposure, or other diseases and disorders associated with the liver. Preferably, the biomarkers are proteins, fragments or derivatives thereof, and are associated with the liver.

In certain inventive embodiments, the biomarkers are liver proteins, peptides, fragments or derivatives thereof which are detected by an assay. An inventive in vitro diagnostic device further includes a process for determining the liver injury of a subject by measuring a sample obtained from the subject at a first time for a quantity of a first biomarker selected from the group of Argininosuccinate synthetase (ASS), argininosuccinate lyase (ASL), sulfuration (estrogen sulfotransferase (EST), SULT2A1, squalene synthase (SQS), liver glycogen phosphorylase (GP), carbamoyl-phosphate synthetase (CPS-1), α-enolase 1, glucose-regulated protein (GRP), ALT, LDH or spectrin breakdown products. The sample is also measured for a quantity of at least one additional liver biomarker. Through comparison of the quantity of the first biomarker and the quantity of the at least one additional liver biomarker to normal levels for each biomarker, the liver injury of the subject and its severity is determined. A ratio is readily calculated of the concentration of two or more biomarkers collected from a sample at a given time. The ratio is then compared with concentration of the two or more biomarkers at a later time to provide clinically relevant information such as the type of hepatic tissues injured, severity of injury, the effectiveness of a therapy, or a combination thereof. It is appreciated that the biomarker data of the present invention is readily supplemented with conventional data such as sonogram data, CT scan data, MRI scan data, and combinations thereof.

An inventive in vitro diagnostic device necessarily incorporates an assay for determining the liver injury of a subject is also provided. The assay includes at least a first biomarker specifically binding agent wherein a first biomarker is one of ASS, EST, EST-1, CPS-1, SULT2A1, SQS, GP, GRP, ALT, LDH or SBDP's. In certain inventive embodiments, an assay is incorporated which may detect one or more markers selected from the group of ASS, EST, CPS-1, SULT2A1, or ALT.

Other aspects of the invention are described infra.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIG. 1 is a schematic view of the in vitro diagnostic device.

FIG. 2 is a series of blots showing hepatic levels of (A) aII-spectrin, (B) argininosuccinate synthase, and (C) γ-GTP in intact, ischemia/reperfusion and intact liver treated in vitro with caspase-3 or calpain-2. (A) shows detection of all-spectrin. (B) shows detection of argininosuccinate synthetase (ASS). (C) shows detection of γ-GTP.

FIG. 3 is a blot showing detection of EST-1 in the liver, serum and plasma after 30 minutes of ischemia.

FIG. 4 is a blot showing the detection of glutathione-S-transferase in rat plasma, using ECL detection methods. Liver injuries included in the experiments were: Ale-chronic alcohol treatment in rats. I/R-liver ischemia/reperfusion. Controls were: S-sham operated rats, no vena portae ligation. N-nalve, intact rats. (normal rats). RC1-high sucrose Alc control. M-molecular weight markers.

FIG. 5 is a blot showing the detection of γ-GTP in rat plasma, using ECL detection methods. Liver injuries included in the experiments were: Alc-chronic alcohol treatment in rats. I/R-liver ischemia/reperfusion. Controls were: S-sham operated rats, no vena portae ligation. N-naive, intact rats. (normal rats). RC1-high sucrose Alc control. M-molecular weight markers.

FIG. 6 is a blot showing the detection of ALT in rat plasma, using ECL detection methods. Liver injuries included in the experiments were: Alc—chronic alcohol treatment in rats. I/R-liver ischemia/reperfusion. Controls were: S-sham operated rats, no vena portae ligation. N-naive, intact rats. (normal rats). RC1-high sucrose Alc control.M-molecular weight markers.

FIG. 7 is a Western blot showing accumulation of biomarkers of liver injury in blood after hepatic ischemia/reperfusion, chronic alcoholic disease and acute endotoxic liver injury.

FIG. 8 (A) shows an increase in serum ASS after repeated i.p. injection of Ecstasy (MDMA) after injection of a total of 40 mg/kg. (B) shows an increase in serum SULT2A1 after repeated i.p. injection of MDMA after injection of a total of 40 mg/kg.

### DETAILED DESCRIPTION OF THE PREFERRED EMBODIMENTS

The present invention has utility in the diagnosis and management of liver injury, liver damage, and liver disease. Through the measurement of biomarkers such as ASS, EST, EST-1, CPS-1, SULT2A1, SQS, GP, GRP, ALT, LDH or SBDP's from a subject alone or in combination, a determination of subject liver injury is provided with greater specificity than previously attainable. The present description is directed toward a first biomarker of ASS for illustrative purposes only and is not meant to be a limitation on the practice or scope of the present invention. It is appreciated that the invention encompasses several other first and additional biomarkers illustratively including EST, CPS-1, SULT2A1, or ALT. The description is appreciated by one of ordinary skill in the art as fully encompassing all inventive biomarkers as an inventive first biomarker as described herein. Surprisingly, by combining the detection of more than one biomarker, a synergistic result is achieved. Illustratively, combining the detection of two neuroactive biomarkers such as ASS and EST provides sensitive detection that is unexpectedly able to discern the level and severity of a liver injury in a subject.

The present invention further incorporates by reference the disclosures presented in US 2006/0246489 and US 2010/0196942. The in vitro diagnostic devices described herein have incorporated assays contained therein, which assays may be substituted herein using the methods therein contained.

The present invention provides for the detection of a liver in jury, liver damage, or liver disease in patients at risk of developing liver damage. Patients “at risk of developing liver damage” include those patients who are anticipated to be exposed to or who have been exposed to any factor known to have the potential of inducing liver damage. This includes exposure to hepatotoxic compounds (whether as part of a therapy or due to accidental exposure), in doses conventionally considered safe or in doses conventionally considered unsafe, radiation, or any clinical therapy useful in the treatment of a disease, wherein said clinical therapy is known to induce liver damage. The definition further includes actual or potential sustained liver injury through physical trauma including, blunt trauma, gunshot wounds, or surgery. Patients at risk of developing liver damage include those patients having inborn errors of metabolism and who are genetically predisposed to induction of liver damage, or those mammalian patients susceptible to liver damage due to other risk factors including genetic factors, age, sex, nutritional status, exposure to other drugs, and systemic diseases. Patients at risk of developing liver damage also includes those patients who are anticipated to be exposed to or who have been exposed to viruses such as hepatitis A, B, C, D, or E, or autoimmune chronic hepatitis. For the purposes herein, sepsis is also liver injury or liver damage.

In Vitro Diagnostic Device

FIG. 1 schematically illustrates an inventive in vitro diagnostic device. An inventive in vitro diagnostic device includes at least a sample collection chamber 2403, an assay module 2402 used to detect biomarkers of neural injury or neuronal disorder, and a user interface that relates the amount of the measured biomarker measured in the assay module. The in vitro diagnostic device may include of a handheld device, a bench top device, or a point of care device.

The sample chamber 2403 can be of any sample collection apparatus known in the art for holding a biological fluid. In one embodiment, the sample collection chamber can accommodate any one of the biological fluids herein contemplated, such as whole blood, plasma, serum, urine, sweat or saliva.

The assay module 2402 is, in certain inventive embodiments, an assay which may be used for detecting a protein antigen in a biological sample, for instance, through the use of antibodies in an immunoassay. The assay module 2402 includes any assay currently known in the art; however the assay should be optimized for the detection of neural biomarkers used for detecting neural injury or neuronal disorder in a subject. The assay module 2402 is in fluid communication with the sample collection chamber 2403. In one embodiment, the assay module 2402 is an immunoassay where the immunoassay may be any one of a radioimmunoassay, ELISA (enzyme linked immunosorbent assay), “sandwich” immunoassay, immunoprecipitation assay, precipitin reactions, gel diffusion precipitin reactions, immunodiffusion assay, fluorescent immunoassay, chemiluminescent immunoassay, phosphorescent immunoassay, or an anodic stripping voltammetry immunoassay. In one embodiment a colorimetric assay may be used which may include only of a sample collection chamber 2403 and an assay module 2402 of the assay. Although not specifically shown these components are preferably housed in one assembly 2407. The assay module 2402 may contain additional agents to detect additional biomarkers, as is described herein.

In a preferred embodiment, the inventive in vitro diagnostic device contains a power supply 2401, an assay module 2402, a sample chamber 2403, and a data processing module 2405. The power supply 2401 is electrically connected to the assay module and the data processing module. The assay module 2402 and the data processing module 2405 are in electrical communication with each other. As described above, the assay module 2402 may include any assay currently known in the art; however the assay should be optimized for the detection of neural biomarkers used for detecting neural injury or neuronal disorder in a subject. The assay module 2402 is in fluid communication with the sample collection chamber 2403. The assay module 2402 includes an immunoassay where the immunoassay may be any one of a radioimmunoassay, ELISA (enzyme linked immunosorbent assay), “sandwich” immunoassay, immunoprecipitation assay, precipitin reactions, gel diffusion precipitin reactions, immunodiffusion assay, fluorescent immunoassay, chemiluminescent immunoassay, phosphorescent immunoassay, or an anodic stripping voltammetry immunoassay. A biological sample is placed in the sample chamber 2403 and assayed by the assay module 2402 detecting for a biomarker of neural injury or neuronal disorder. The measured amount of the biomarker by the assay module 2402 is then electrically communicated to the data processing module 2404. The data processing 2404 module may include of any known data processing element known in the art, and may include of a chip, a central processing unit (CPU), or a software package which processes the information supplied from the assay module 2402.

In one embodiment, the data processing module 2404 is in electrical communication with a display 2405, a memory device 2406, or an external device 2408 or software package (such as laboratory and information management software (LIMS)). In one embodiment, the data processing module 2404 is used to process the data into a user defined usable format. This format includes the measured amount of liver biomarkers detected in the sample, indication that liver injury or liver damage is present, or indication of the severity of liver injury or liver damage. The information from the data processing module 2404 may be illustrated on the display 2405, saved in machine readable format to a memory device, or electrically communicated to an external device 2408 for additional processing or display. Although not specifically shown these components are preferably housed in one assembly 2407. In one embodiment, the data processing module 2404 may be programmed to compare the detected amount of the biomarker transmitted from the assay module 2402, to a comparator algorithm. The comparator algorithm may compare the measure amount to the user defined threshold which may be any limit useful by the user. In one embodiment, the user defined threshold is set to the amount of the biomarker measured in control subject, or a statistically significant average of a control population.

In one embodiment, an in vitro diagnostic test may include one or more devices, tools, and equipment configured to hold or collect a biological sample from an individual. In one embodiment of an in vitro diagnostic test, tools to collect a biological sample may include one or more of a swab, a scalpel, a syringe, a scraper, a container, and other devices and reagents designed to facilitate the collection, storage, and transport of a biological sample. In one embodiment, an in vitro diagnostic test may include reagents or solutions for collecting, stabilizing, storing, and processing a biological sample. Such reagents and solutions for nucleotide collecting, stabilizing, storing, and processing are well known by those of skill in the art and may be indicated by specific methods used by an in vitro diagnostic test as described herein. In another embodiment, an in vitro diagnostic test as disclosed herein, may include a micro array apparatus and reagents, a flow cell apparatus and reagents, a multiplex nucleotide sequencer and reagents, and additional hardware and software necessary to assay a genetic sample for certain genetic markers and to detect and visualize certain biological markers.

### Data Processing Module

FIG. 1 further illustrates a data processing module 2404 contained within the in vitro diagnostic device. The data processing module 2404 includes of a central processing unit (CPU), a memory unit, an input/output component, and a network component. The data processing module 2404 receives instructions from software to process data received from the assay module 2402 into a user defined usable format. The information generated from the data processing module 2404 may be illustrated on the display 2405 includes a graphical user interface (GUI), saved in machine readable format to the memory unit, or electrically communicated to an external device 2408 for additional processing or display by wired or wireless communication. The CPU carries out the software's instructions and dictates the data processing module's remaining components to process any inputs and signals received from the assay module 2402.

The input component receives a signal, an input, from the assay model 2402 stating the measured amount of a specific biomarker present in an analyzed sample. The data processing module 2404 receives and compares the input to a preprogrammed threshold level, predetermined for each biomarker respectively. The result of the comparison is a determination of the presence and severity of liver injuries. The results may be saved to the memory unit for later access by the user. After the comparison, the data processing module 2404 generates an output signal of the processed measure amount based on the comparison of the input to a preprogrammed threshold.

The memory unit stores the data containing a preprogrammed threshold level for each respective biomarker. The data processing module 2404 accesses the preprogrammed threshold level to compare an input to a biomarker's proper levels. The preprogrammed threshold level is a predetermined amount based on a known positive level. In an alternative embodiment, the preprogrammed threshold may be a predetermined amount based on a known negative level. In an alternative embodiment, the preprogrammed threshold level may be an amount of the biomarker measured in normal control. The specific level for each of the embodiments is determined through prior experimentation. The memory unit also stores the results of the data processing module's comparison.

The output component relays to the display 2405 the processed measured amount, resulting from the comparison of the input to the preprogrammed threshold, in a user defined usable format. The display 2405 provides an output from which the user may determine the measured amount of the respective biomarker present in the sample, an indication of the presence or absence of liver injuries, and/or an indication of the severity of liver injuries.

The network component electronically communicates the processed data through a wired connection to an external device at a remote location. The user may directly connect the in vitro diagnostic device to another computer to download the data stored to be saved in a separate location for additional processing or display. In an alternative embodiment, the network component may consist of a wireless feature. The wireless feature allows the user to transfer the processed data to an external device at a remote location without the need of a direct connection.

In an alternative embodiment, the data processing module 2404 may compare the levels of two or more biomarkers to determine the type of liver injury present. The input component receives multiple inputs from a multiplex assay module. The data processing module 2404 then compares each signal to the respective biomarker's threshold level. The output component relays the processed measured amounts, resulting from the comparison, to the display 2405. The display 2405 provides an output from which the user may determine the measured amount of the respective biomarker present in the sample, an indication of the presence or absence of liver injuries, and/or an indication of the severity of liver injuries. Additionally, through the comparison of measure amounts of multiple biomarkers, the data processing module 2404 may generate data from which the user may determine the specific type of liver injury. The output component may also relay this data to the display 2405.

Liver Biomarker's

In a preferred embodiment, detection the inventive in vitro diagnostic device provides the ability to detect and monitor levels of proteins after liver damage. One or more enzymes of arginine/urea/nitric oxide cycle, sulfuration enzymes and spectrin breakdown related products is diagnostic of liver injury. Examples of these markers include, but not limited to: argininosuccinate synthetase (ASS) and argininosuccinate lyase (ASL), sulfuration (estrogen sulfotransferase (EST), squalene synthase (SQS), liver glycogen phosphorylase (GP), carbamoyl-phosphate synthetase (CPS-I), α-enolase 1, glucose-regulated protein (GRP) and spectrin breakdown products.

In another preferred embodiment, detection of one or more biomarkers can be correlated to known diagnostic tests of liver injury. Examples include: liver function tests—assessment of hepatic clearance of organic anions, such as, bilirubin, indocyanine green (ICG), sulfobromophthalein (BSP) and bile acids; assessment of hepatic blood flow by measurements of galactose and ICG clearance; and assessment of hepatic microsomal function, through the use of the aminopyrine breath test and caffeine clearance test.

In certain inventive embodiments, detection of the biomarkers is diagnostic of liver injury. Liver injury is a result of any factors. For example, liver ischemic injury; liver damage induced by hepatotoxic compounds including direct cytotoxicity including drug hypersensitivity reactions, cholestasis, and injury to the vascular endothelium (Sinclair et ah, Textbook of Internal Medicine, 569-575 (1992) (editor, Kelley; Publisher, J. B. Lippincott Co.).

It is appreciated however, that multiple biomarkers may be predictors of different modes or types of liver injury or liver damage to the same cell type. Through the use of an inventive assay inclusive of biomarkers associated with the liver as well as at least one other type of liver biomarker, the type of liver cells being stressed or killed as well as quantification of the liver injury or liver damage provides rapid and robust diagnosis of liver injury or liver damage.

### Enzyme Deficiencies

In certain inventive embodiments, lack of detection (i.e. absence) of liver enzymes, e.g. ASS, is diagnostic of liver enzyme diseases. For example, lack of ASS (Argininosuccinate Synthetase Deficiency) is a genetic disease: Maple Syrup Urine Disease (MSUD) and Citrullinemia. Baseline levels in healthy controls are detectable with the methods of the invention and would expect to see below normal values in humans affected by the condition. In one embodiment, the compositions and methods of the invention identify at risk individuals. The identification can be determined in families, pregnant females by extracting samples such as blood, serum, amniotic fluid and the like. This would allow identification of risk and/or diagnosis of disease in an infant or fetus.

In certain inventive embodiments, detection of the absence of one or more enzymes of arginine/urea/nitric oxide cycle, sulfuration enzymes and spectrin breakdown related products is diagnostic of liver enzyme deficiency associated diseases. Examples of these markers include, but not limited to: argininosuccinate synthetase (ASS) and argininosuccinate lyase 24 (ASL), sulfuration (estrogen sulfotransferase (EST), squalene synthase (SQS), liver glycogen phosphorylase (GP), carbamoyl-phosphate synthetase (CPS-1), α-enolase 1, glucoseregulated protein (GRP) and spectrin breakdown products.

### Biological Samples

Biological samples of blood, urine and saliva are collected using normal collection techniques. For example, and not to limit the sample collection to the procedures containted herein, blood samples may be collected by venipuncture in Vacutainer tubes, and if preferred spun down and separated into serum and plasma. For Urine and saliva, samples are collected avoiding the introduction of contaminants into the specimen is preferred. All biological samples may be stored in aliquots at −80° C. for later assay. Surgical techniques for obtaining solid tissue samples are well known in the art. Any suitable biological samples can be obtained from a subject to detect markers. It should be appreciated that the methods employed herein may be identically reproduced for any biological fluid to detect a marker or markers in a sample.

After insult, the damaged tissue or organs in in vitro culture or in situ in a subject express altered levels or activities of one or more proteins than do such cells not subjected to the insult. A biological sample including cells or fluid secreted from these cells might also be used in an adaptation of the inventive methods to determine and/or characterize an injury to such non-liver cells.

Baseline levels of several biomarkers are those levels obtained in the target biological sample in the species of desired subject in the absence of a known liver condition. These levels need not be expressed in hard concentrations, but may instead be known from parallel control experiments and expressed in terms of fluorescent units, density units, and the like. Typically, baselines are determined from subjects where there is an absence of a biomarker or present in biological samples at a negligible amount. However, some proteins may be expressed less in an injured patient. Determining the baseline levels of protein biomarkers in a particular species is well within the skill of the art. Similarly, determining the concentration of baseline levels of liver injury biomarkers is well within the skill of the art.

### Immunoassays

Antibodies directed against anyone of the liver biomarkers (e.g., argininosuccinate synthetase (ASS) and argininosuccinate lyase (ASL), sulfuration (estrogen sulfotransferase (EST), squalene synthase (SQS), liver glycogen phosphorylase (GP), carbamoyl-phosphate synthetase (CPS-I), a-enolase 1, glucose-regulated protein (GRP) and spectrin breakdown products) can be used, as taught by the present invention, to detect and diagnose liver injury disease. Various histological staining methods, including immunohistochemical staining methods, may also be used effectively according to the teaching of the invention.

The inventive in vitro diagnostic device makes use of an assay module 402, which may be one of many types of assays. The biomarkers of the invention can be detected in a sample by any means. Methods for detecting the biomarkers are described in detail in the materials and methods and Examples which follow. For example, immunoassays, include but are not limited to competitive and non-competitive assay systems using techniques such as western blots, radioimmunoassays, ELISA (enzyme linked immunosorbent assay), “sandwich” immunoassays, magnetic immunoassays, radioisotope immunoassay, fluorescent immunoassays, immunoprecipitation assays, precipitin reactions, gel diffusion precipitin reactions, immunodiffusion assays, fluorescent immunoassays, chemiluminescent immunoassays, phosphorescent immunoassays, anodic stripping voltammetric immunoassay and the like. Inventive in vitro diagnostic devices may also include any know devices currently available that utilize ion-selective electrode potentiometry, microfluids technology, fluorescence or chemiluminescence, or reflection technology that optically interprets color changes on a protein test strip. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation). It should be appreciated that at present none of the existing technologies present a method of detecting or measuring any of the ailments disclosed herein, nor does there exist any methods of using such in vitro diagnostic devices to detect any of the disclosed biomarkers to detect their associated injuries.

As used herein the term “diagnosing” means recognizing the presence or absence of a neurological or other condition such as an injury or disease. Diagnosing is optionally referred to as the result of an assay wherein a particular ratio or level of a biomarker is detected or is absent.

As used herein a “ratio” is either a positive ratio wherein the level of the target is greater than the target in a second sample or relative to a known or recognized baseline level of the same target. A negative ratio describes the level of the target as lower than the target in a second sample or relative to a known or recognized baseline level of the same target. A neutral ratio describes no observed change in target biomarker.

As used herein an injury is an alteration in cellular or molecular integrity, activity, level, robustness, state, or other alteration that is traceable to an event. Injury illustratively includes a physical, mechanical, chemical, biological, functional, infectious, or other modulator of cellular or molecular characteristics. An event is illustratively, a physical trauma such as an impact (percussive) or a biological abnormality such as a stroke resulting from either blockade or leakage of a blood vessel. An event is optionally an infection by an infectious agent. A person of skill in the art recognizes numerous equivalent events that are encompassed by the terms injury or event.

An exemplary process for detecting the presence or absence of a biomarker, alone or in combination, in a biological sample involves obtaining a biological sample from a subject, such as a human, contacting the biological sample with a compound or an agent capable of detecting of the marker being analyzed, illustratively including an antibody or aptamer, and analyzing binding of the compound or agent to the sample after washing. Those samples having specifically bound compound or agent express the marker being analyzed.

For example, in vitro techniques for detection of a marker illustratively include enzyme linked immunosorbent assays (ELISAs), radioimmuno assay, radioassay, western blot, Southern blot, northern blot, immunoprecipitation, immunofluorescence, mass spectrometry, RT-PCR, PCR, liquid chromatography, high performance liquid chromatography, enzyme activity assay, cellular assay, positron emission tomography, mass spectroscopy, combinations thereof, or other technique known in the art. Furthermore, in vivo techniques for detection of a marker include introducing a labeled agent that specifically binds the marker into a biological sample or test subject. For example, the agent can be labeled with a radioactive marker whose presence and location in a biological sample or test subject can be detected by standard imaging techniques. Optionally, the first biomarker specifically binding agent and the agent specifically binding at least one additional neuroactive biomarker are both bound to a substrate. It is appreciated that a bound agent assay is readily formed with the agents bound with spatial overlap, with detection occurring through discernibly different detection for first biomarker and each of at least one additional neuroactive biomarkers. A color intensity based quantification of each of the spatially overlapping bound biomarkers is representative of such techniques.

Any suitable molecule that can specifically bind to a biomarker and any suitable molecule that specifically binds one or more other biomarkers of a particular condition are operative in the invention to achieve a synergistic assay. In certain inventive embodiments, an agent for detecting the one or more biomarkers of a condition is an antibody capable of binding to the biomarker being analyzed. In certain inventive embodiments, an antibody is conjugated with a detectable label. Such antibodies can be polyclonal or monoclonal. An intact antibody, a fragment thereof (e.g., Fab or F(ab′)₂), or an engineered variant thereof (e.g., sFv) can also be used. Such antibodies can be of any immunoglobulin class including IgG, IgM, IgE, IgA, IgD and any subclass thereof. Antibodies for numerous inventive biomarkers are available from vendors known to one of skill in the art. Illustratively, antibodies directed to inventive biomarkers are available from Santa Cruz Biotechnology (Santa Cruz, Calif.). Exemplary antibodies operative herein are used to detect a biomarker of the disclosed conditions. In addition antigens to detect autoantibodies may also be used to detect chronic injury of the stated injuries and disorders.

An antibody is optionally labeled. A person of ordinary skill in the art recognizes numerous labels operable herein. Labels and labeling kits are commercially available optionally from Invitrogen Corp, Carlsbad, Calif. Labels illustratively include, fluorescent labels, biotin, peroxidase, radionucleotides, or other label known in the art. Alternatively, a detection species of another antibody or other compound known to the art is used as form detection of a biomarker bound by an antibody.

Antibody-based assays are used in certain inventive embodiments for analyzing a biological sample for the presence of a biomarker and one or more other biomarkers of a particular injury or condition. Suitable western blotting methods are described below in the examples section. For more rapid analysis (as may be important in emergency medical situations), immunosorbent assays (e.g., ELISA and RIA) and immunoprecipitation assays may be used. As one example, the biological sample or a portion thereof is immobilized on a substrate, such as a membrane made of nitrocellulose or PVDF; or a rigid substrate made of polystyrene or other plastic polymer such as a microtiter plate, and the substrate is contacted with an antibody that specifically binds one of the other biomarkers under conditions that allow binding of antibody to the biomarker being analyzed. After washing, the presence of the antibody on the substrate indicates that the sample contained the marker being assessed. If the antibody is directly conjugated with a detectable label, such as an enzyme, fluorophore, or radioisotope, the presence of the label is optionally detected by examining the substrate for the detectable label. Alternatively, a detectably labeled secondary antibody that binds the marker-specific antibody is added to the substrate. The presence of detectable label on the substrate after washing indicates that the sample contained the marker.

Numerous permutations of these basic immunoassays are also operative in the invention. These include the biomarker-specific antibody, as opposed to the sample being immobilized on a substrate, and the substrate is contacted with a biomarker conjugated with a detectable label under conditions that cause binding of antibody to the labeled marker. The substrate is then contacted with a sample under conditions that allow binding of the marker being analyzed to the antibody. A reduction in the amount of detectable label on the substrate after washing indicates that the sample contained the marker.

While antibodies are used in certain inventive embodiments for use in the invention because of their extensive characterization, ther suitable agents (e.g., a peptide, an aptamer, or a small organic molecule) that specifically binds a biomarker is readily used in place of the antibody in the above described immunoassays. For example, an aptamer might be used. Aptamers are nucleic acid-based molecules that bind specific ligands. Methods for making aptamers with a particular binding specificity are known as detailed in U.S. Pat. Nos. 5,475,096; 5,670,637; 5,696,249; 5,270,163; 5,707,796; 5,595,877; 5,660,985; 5,567,588; 5,683,867; 5,637,459; and 6,011,020.

A myriad of detectable labels that are operative in a diagnostic assay for biomarker expression are known in the art. Agents used in methods for detecting a biomarker are conjugated to a detectable label, e.g., an enzyme such as horseradish peroxidase. Agents labeled with horseradish peroxidase can be detected by adding an appropriate substrate that produces a color change in the presence of horseradish peroxidase. Several other detectable labels that may be used are known. Common examples of these include alkaline phosphatase, horseradish peroxidase, fluorescent compounds, luminescent compounds, colloidal gold, magnetic particles, biotin, radioisotopes, and other enzymes. It is appreciated that a primary/secondary antibody system is optionally used to detect one or more biomarkers. A primary antibody that specifically recognizes one or more biomarkers is exposed to a biological sample that may contain the biomarker of interest. A secondary antibody with an appropriate label that recognizes the species or isotype of the primary antibody is then contacted with the sample such that specific detection of the one or more biomarkers in the sample is achieved.

The present invention provides a step of comparing the quantity of one or more biomarkers to normal levels to determine the condition of the subject. It is appreciated that selection of additional biomarkers allows one to identify the types of cells implicated in an abnormal organ or physical condition. The practice of an inventive process provides a test which can help a physician determine suitable therapeutics to administer for optimal benefit of the subject.

The results of such a test using an in vitro diagnostic device can help a physician determine whether the administration a particular therapeutic or treatment regimen may be effective, and provide a rapid clinical intervention to the injury or disorder to enhance a patients recovery.

It is appreciated that other reagents such as assay grade water, buffering agents, membranes, assay plates, secondary antibodies, salts, and other ancillary reagents are available from vendors known to those of skill in the art. Illustratively, assay plates are available from Corning, Inc. (Corning, N.Y.) and reagents are available from Sigma-Aldrich Co. (St. Louis, Mo.).

Methods involving conventional biological techniques are described herein. Such techniques are generally known in the art and are described in detail in methodology treatises such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992.

Various aspects of the present invention are illustrated by the following non-limiting examples. The examples are for illustrative purposes and are not a limitation on any practice of the present invention. It will be understood that variations and modifications can be made without departing from the spirit and scope of the invention. While the examples are generally directed to mammalian tissue, specifically, analyses of mouse tissue, a person having ordinary skill in the art recognizes that similar techniques and other techniques known in the art readily translate the examples to other mammals such as humans. Reagents illustrated herein are commonly cross reactive between mammalian species or alternative reagents with similar properties are commercially available, and a person of ordinary skill in the art readily understands where such reagents may be obtained. Variations within the concepts of the invention are apparent to those skilled in the art.

### EXAMPLES

### Materials and Methods

### Liver Tissue Processing and Sample Preparation.

For high throughput screening—Western blot ((HTS-WB) (PowerBlot)) and conventional Western blot analyses, liver specimens are snap frozen in liquid nitrogen after removal. Liver samples from FR, naïve and sham-operated rats are homogenized on ice using Polytron in RIPA buffer consisting of PBS, 1% Nonidet P-40, 0.5% sodium deoxycholate, 0.1% SDS, 1 mM DTT, containing 0.1 mg/ml PMSF, 1 mM sodium orthovanadate, 5 mM EDTA, 5 mM EGTA and protease inhibitor cocktail (Roche, Inc). For r-caspase-3 and r-calpain-2 treatment in vitro, livers obtained from intact (naïve) rats, are homogenized in RIPA buffer consisting of PBS, 1% Nonidet P-40, 0.5% sodium deoxycholate, 0.1% SDS, 1 mM DTT, 5 mM EDTA, 5 mM EGTA without protease inhibitors. Homogenates are left on ice for 30 min and centrifuged for 15 min at 10,000 rpm at 4° C. Supernatants are removed and protein measured using bicinechoninic acid (Pierce, Inc). Intact liver samples are treated in vitro with caspase-3 (Chemicon, specific act. 1 μg/μl) or calpain-2 (Calbiochem, 0.25 μg/μl).

### High Throughput Screen Western Blot (HTS-WB) and Conventional Western Blot Analyses

The gel is 13×10 cm, 4-15% gradient SDS-polyacrylamide, 0.5 mm thick (Bio-Rad Criterion IPG) 200 μg of protein is loaded in one big well across the entire width of the gel. This translates into ^{˜}8 μg per lane on a standard 10 well mini-gel. The gel is run for 1.5 hours at 150 volts, proteins transferred to Immobilon-P membrane (Millipore). The membrane is blocked for one hour with blocking buffer.

The membrane is clamped with a western blotting manifold that isolates 40 channels across the membrane. In each channel, a complex antibody cocktail is added and hybridized for one hour at 37° C.

The blot is removed, washed and visualized for 30 minutes at 37° C. with secondary goat anti-mouse conjugated to Alexa680 fluorescent dye (Molecular Probes). The membrane is washed, dried and scanned at 700 nm (for monoclonal antibody target detection) using the Odyssey Infrared Imaging System.

MW Standards—Lanes 4 and 40 of all blots are loaded with two standardization cocktails. Data analysis—data analysis includes raw and normalized signal intensity data from each blot with changes greater than 1.5 fold indicated. A description of characteristics of the analysis follow: 1. Quantity—total intensity of a defined spot. 2.

Normalized Quantity—All blots are normalized to the sum intensity of all valid spots on a blot then multiplied by 1,000,000. 3/. Ratio—The Normalized Quantity for experimental bands expressed as a ratio of the Normalized Quantity for the corresponding control bands. The Ratio is used to determine increases or decreases in protein expression. Results are also expressed as Fold Change, a semi-quantitative value that represents the general trend of protein changes, either increasing or decreasing, for the experimental sample relative to control.

Changes are listed in order of confidence, level 10 being the highest confidence. Confidence levels are defined as: a) Level 10—Changes greater than 2 fold in all 4 comparisons from good quality signals that also pass a visual inspection; b) Level 9—Changes 1.5 to 1.9 fold in all 4 comparisons from good quality signals that also pass a visual inspection; c) Level 8—Changes greater than 2 fold in all 4 comparisons from low signals that pass a visual inspection; d) Level 7—Changes 1.25 to 1.5 fold in all 4 comparisons from good quality signals that pass a visual inspection; e) Level 6—Changes greater than 2 fold in all 4 comparisons that do not pass visual inspection; f) Level 5—Changes 1.5 to 1.9 fold in all 4 comparisons that do not pass visual inspection.

### Targeted Analysis of Liver-Specific Proteins

The analysis shown in Table 1, is performed by Western blot using antibody available through various sources. Typically, 25 μg protein are loaded with two identical sets of 5 samples, separated in 4-20% polyacrylamide gel mini-slabs, and transferred onto PVDF membrane. The membrane is cut in 2 pieces, blocked, probed with two different antibodies, visualized using ECL Plus Kit (Pierce, Inc) and scanned. The membranes are stripped using stripping buffer and re-probed with other two antibody. After visualizing the bands with EDCL Plus Kit and scanning, membranes are stripped again, probed with aII-spectrin antibody and developed using alkaline phosphatase detection method. For accurate assessment of molecular mass of developed proteins, two sets of protein standards are added and developed simultaneously (Magic Markers, Invitrogen, Inc).

### Liquid Chromatography, SDS-PAGE, LC/Mass Spectrometry

Briefly, The LC system is set up to run two columns in line: S-sepharose and Q-sepharose. Samples are filtered, protein (1 mg) is loaded into the sample loop, and run using gradient of Mobile Phase A (20 mM Tris-HCl) and B (20 mM Tris-HCl containing 1M NaCl. Fractions (1 ml) are collected, 1 fraction per minute, for a total of 32 fractions. The fractions are concentrated and subjected to SDS-PAGE on BioRad Criterion Gels, 4-20% Tris-HCl 18 well gels. The samples are run in pairs: sham-operated (control); I/R; untreated in vitro (control in vitro); caspase-3- and/or calpain-2 treated next to each other for each fraction. Gels are stained with Coomassie-R250 and are used to select bands for excision.

Band excision, protein reduction, alkylation, digestion and extraction is performed as previously described (Wang, K. K., Ottens, A., Haskins, W., Liu, M. C., Kobeissy, F., Denslow, N., Chen, S., and Hayes, R. L. (2004) Proteomics studies of traumatic brain injury. Int Rev Neurobiol 61, 215-240)). The LC which is used to elute the peptides from the column has three phases: Mobile Phase A—99.6% water, 0.4% acetic acid; Mobile Phase B—the organic phase-20% water, 0.4% acetic acid, 79.6% Methanol; Mobile Phase C—used for loading the sample from the tube to the column is 0.4% acetic acid, 4% acetonitrile, and 95.6% water. For mass-spectrometry, samples reconstituted in 15 μL of Mobile Phase C solution.

The MS is a LCQ Deca XP, quadrapole ion trap mass spectrometer. The peptides are loaded on to a reverse phase column and eluted into the MS using an organic gradient and electrospray ionization. Once the ions are inside the MS, several scans take place. First the full MS scan—every mass to charge value (m/z) from the sample that has entered the ion trap at the time of the scan is recorded. Each peak represents a mass to charge value which represents the parent ion. The mass spec picks the three most intense parent ions and does another scan, the MSMS scan. Each parent ion fragments into a product ion which produces spectra unique to a peptide. So, for each MS scan, three MSMS spectra are produced, each likely representing a different peptide. The collection of all these scans plotted together is the chromatogram, which is send to BioWorks Browser. When a spectrum in the sample matches a spectrum in the database, it is assigned an Xcorr. This value indicates the level of similarity of the two spectra.

### Liver Sample Preparation, Protein Expression Studies and Analysis

These are performed by conventional Western blot. The antibody used for a II-spectrin determination is against full size aII-spectrin molecule (Affinity, Inc), caspase-3 cleaved 150i fragment (Cell Signalling Technologies), caspase-3 generated 120 kDa fragment (Abs from our laboratory-CNPBR-UF) and calpain-2 cleaved 150 fragment (CNPBR-UF). Antibody against hepatic biomarkers is obtained from various commercial and non-commercial sources.

### Serum and Plasma Sample Preparation

Blood is collected from rat heart at the end of experimental procedures. Plasma is obtained from K-EDTA preserved blood by centrifugation. 20 μl of plasma or serum is mixed with 180 μl of RIPA buffer (with proteases inhibitors), vortexed, incubated on ice for 30 min and centrifuged. Supernatants are removed, aliquots mixed with sample buffer 1:1, heated and loaded onto gel.

### Serum Enzyme Assays

ALT, LDH and γ-GTP activities are determined using kinetic methods with commercial Kits according to manufacturer's instructions.

### Liver Immunohistochemistry

Liver specimens of experimental rats is taken at the end of reperfusion for analysis of tissue injury. Samples of liver tissue are placed in 10% neutral formaline for routine H&E staining according to a standard protocol, or frozen immediately in OCT buffer for immunohistochemistry.

### Immunostaining of Activated Caspase 3 and Calpain-2

Cryopreserved 4 μm liver frozen sections are fixed in ice-cold 4% paraformaldehyde in PBS or −20° C. methanol for 20 min on ice. Samples are washed 3 times with PBS for 3 minutes each, permeabilized on ice with cold 0.5% Triton X-100/PBS/0.2% sucrose, washed with PBS and quenched with 0.1% sodium borohydride for 5 min. Samples are blocked at room temperature (RT) for 30 minutes in 20% goat serum in PBS and incubated overnight (+40° C.) in 20% goat serum/PBS with activated caspase-3 (17/19 kDa protein, Cell Signaling, Inc.) or calpain-2 (Chemicon, Inc.) mouse monoclonal antibody. After extensive wash, cover slips are incubated with anti-mouse IgG conjugated with fluorescent dye (AlexaGreen 488, Molecular Probes). The cover slips are mounted and analyzed using fluorescent microscope equipped with the Optical Camera (Zeiss, Inc).

### In Situ TUNNEL Assays on Liver Tissue Section

TUNNEL Assays are performed using commercial PROMEGA Kit. Liver samples are fixed in 10% buffered formalin and embedded in paraffin. Tissue sections are placed on slides and then deparaffinized and rehydrated. Slides are subjected to proteinase K digestion for 15 minutes (0.2 M Tris/0.5 M EDTA, pH 8.0, proteinase K 1 mg/ml). Slides are washed with PBS, equilibrated with buffer for 15 min and stained with FITC-conjugated nucleotide mixture and TdT for 80 minutes. The enzymatic reaction is stopped, slides are counterstained with a propidium iodide/anti-fade DNA solution and photographed using fluorescent microscope equipped with the Optical Camera (Zeiss, Inc) with appropriated filter.

### ASS Antibodies

A monoclonal antibody against the C′ terminus of ASS is commercially available (BD Transduction Laboratories). Additional monoclonal antibodies are being produced in the Hybridoma Core Laboratory in the Biotechnology Program at the University of Florida. Mice are immunized with the same materials that are used to prepare the specific anti-ASS polyclonal antibody in rabbits. Hybridomas producing the desired monoclonal antibodies are cloned two times to ensure their stability and purity. At least 100 aliquots of founder cloned hybridoma cells are frozen to ensure a life-long supply of the antibody.

### Antibody Analysis

Antigen binding affinities of all ASS antibodies are analyzed using indirect ELISA, Western blots, and the BIAcore 3000. For each specific ELISA capture and detection antibody pairs are selected that give optimal antigen binding and affinity. The selection of antibodies is based on those antibodies that have high affinities and recognize different epitopes on the biomarkers. Antibodies are selected that have high affinity constants composed of a fast on rate and a slow off rate as determined by the BIAcore 3000 (Protein Chemistry Core at the University of Florida). The BIAcore is a chip-based device that allows determination of affinity constants (including on and off rates) of the interactions between proteins and other proteins, peptides, or DNA. The instrument uses a highly sensitive surface plasmon resonance detection system allowing precise determination of affinity constants in real time without addition of exogenous labels.

### Production of Recombinant ASS Antigen and Validation of SW ELISA

ASS cDNA is commercially available from ATCC and is used for the production of recombinant protein. For standard curve assay, a serial dilution of 50-0.001 ng of purified protein/well are analyzed. This deter wines the dynamic range of the assay which is anticipated to be 100-1000-fold, encompassing concentrations that are likely to be present in blood or serum. The SW ELISA data have shown that the sensitivity is greater than highly sensitive ECL Western blot (high pg level)

### BIAcore:

Two pmol of biomarker protein (50 ng) in PBST is diluted in 10 mM acetate buffer (pH 4.5). FC1 is of a CM5 chip is used as control, whereas FC2 is activated and injected with 5 μl biomarker at 10 μl/min, yielding a ΔRU of 475. Antibodies are diluted 1:10 in PBST and 20 μl are injected for 2 min (kinject) followed by dissociation of 3 min. Regeneration of the surface is performed by injecting 5 μL glycine (10 mM, pH 1.5). Antibody injection and regeneration is repeated without loss of surface reactivity.

### Example 1

### Expression of Argininosuccinate Synthase (ASS)

The data show that the ASS protein is expressed in adult human tissues in the liver, much lesser extent kidney, and at very low levels, in testes. (i) ASS and its caspase-3 mediated breakdown products are up-regulated in the liver, and (ii) ASS accumulated in plasma after 30 min liver ischemia followed by 30 mM reperfusion. A number of experiments are conducted using a human model of ischemia/reperfusion with the particular emphasis on different reperfusion time with a fixed 30 min of ischemia period. Accumulation of ASS in blood is time-dependent and attained a steady state at 3 hours after reperfusion. Plasma ASS levels correlated strongly with the severity of hepatic damage determined by classical histology analysis of liver tissue and immunostaining with activated caspase-3.

### Example 2

### Sandwich ELISA for the Specific and Quantitative Detection of Argininosuccinate Synthase (ASS) in Biological Fluids

Based on human ASS protein published sequence (P00966), two peptides are designed and synthesized: N-47-ARKKALKLGAKKV-59-C (SEQ ID NO: 1) and N-2,4-AKAPNTPDILEIEFKK-229-C (SEQ ID NO: 2). Currently, these peptides are employed to produce rabbit polyclonal antibody.

The sandwich ELISA assay is used as a diagnostic for liver ischemic injury in humans. This includes liver transplantation, acute liver failure of various etiology, septic shock due to abdominal and multiple trauma. Data show that similarly to rats, plasma ASS is not detected in control, healthy persons.

The ELISA is normalized against a measurement of known amounts of ASS in biological fluids, such as serum and plasma and tissues including liver. The standard ASS is obtained as recombinant GST-tagged protein. It is determined that the ASS monoclonal antibody recognized ASS at 46 kDa with high specificity and sensitivity. This antibody is used as a detection antibody in the specific ELISA. The produced two rabbit polyclonal antibodies that are specific for ASS are tested using BIAcore and the concentration is optimized for use in the ELISA by varying the concentration of antibody and ASS standard in controlled titration experiments. Various concentrations of protein and antibody are tested to determine the specificity and sensitivity of the antibody. Concentrations of protein and antibody that give 80% of the highest binding are chosen for the sandwich ELISA.

To determine reactivity and specificity of the antibodies a tissue panel is probed by Western blot. An indirect ELISA method is used with the recombinant ASS protein attached to the ELISA plate to determine the optimal concentrations of the antibodies to be used in the assay. This assay determines the robust concentration of anti-ASS to use in the assay. 96-well microplates are coated with 50 ng/well and the rabbit and mouse anti-Ass antibodies are serially diluted starting with a 1:250 dilution down to 1:10,000 to determine the optimum concentration to use for the assay. A secondary anti-rabbit (or mouse)-horseradish peroxidase (HRP) labeled detection antibody and Ultra-TMB are used as detection substrates to evaluate the results.

Once the concentration of antibody is determined for maximum signal, the maximum detection limit of the indirect ELISA for each antibody is determined. 96-well microplate are coated from 50 ng/well serially diluted to <1 pg/well. For detection the antibodies are diluted to the concentration determined above. This provides a sensitivity range for the ASS ELISA assays and the choice of antibody for capture and detection.

To optimize and enhance the signal in the sandwich ELISA, the detection antibody is directly labeled with HRP to avoid any cross reactivity and to enhance the signal with the amplification system, which is very sensitive. This format and amplification has successfully worked for other biomarkers in our laboratory. To build the SW ELISA assay, the wells of the 96-well plate are coated with saturating concentrations of purified antibody (250 ng/well), the concentration of ASS antigen will range from 50 ng to <1 pg/well and the detection antibody will be at the concentration determined above. Initially the complex is detected with a HRP-labeled secondary antibody to confirm the SW ELISA format, but will replace the detection system by the HRP-labeled detection antibody.

### Example 3

### Identification of Altered Proteins and their Breakdown Products in Human Liver

Rats (Sprague Dawley, male, 225-250 g) are anesthetized with isofluoran, hepatoduodenal ligament immobilized and hepatic triad (portal vein, hepatic artery and bile duct) is occluded with small vascular clamp for 30 min of normothermic ischemia followed by 30 min reperfusion. At the end, blood is withdrawn; liver is briefly perfused with ice-cold PBS and removed for analysis. Sham operated rats are subjected to anesthesia without ligation of hepatic triad. Intact liver tissue and blood are obtained immediately after rat anesthesia. Intact liver tissues are treated in vitro with recombinant caspase-3 or calpain-2. Initially, for I/R injury biomarker discovery, a custom mini-array of 40 antibodies is designed.

The results are presented as images of 40 antibody western blot mini-screen of control (sham-operated) and I/R samples, in vitro caspase-3-treated intact samples and calpain-2 treated samples.

### Example 4

### Characterization of Novel Hepatic Biomarkers of Liver Injury

Hepatic all spectrin is cleaved in I/R liver via both caspase-3 and calpain-2 with accumulation of SBDP 150 kDa and 120 kDa (FIG. 3A). Characterization and comparison of three hepatic proteins as potential biomarkers of I/R-induced liver injury, included the following: argininosuccinate synthase (AS), liver isoform glutathione-S-transferase (GST-BB), and also γ-GTP and ALT, classic markers of hepatocellular injury. AS and γ-GTP are examined in liver tissues using western blot analysis with a particular emphasis on accumulation of possible breakdown products via caspase-3 and/or calpain-2 (FIGS. 3B and 3C).

As seen in FIG. 3C, a major band of γ-GTP in the liver (140 kDa) is different from a predicted M.W. of 90 kDa, and appears to be modified in I/R liver similarly to caspase-3 treated livers with additional accumulation of ^{˜}70 kDa minor immunoreactivity. Preliminary validation of diagnostic values of novel hepatic biomarkers is performed in blood plasma.

It has been found that intact ASS protein (46 kDa) accumulated in plasma of rats subjected to 30/30 min ischemia/reperfusion, but it is absent in plasma from normal or sham-operated rats and in rats with chronic alcoholic administration. Surprisingly, there are no ASS cleavage fragments in I/R plasma contrary to what is observed in liver tissue at this time point (FIG. 3 B). Very low levels of GST-BB, hepatic isoform of GST, are detectable in normal rat plasma as ^{˜}51 kDa protein (predicted M.W.-23-25 kDa). GST-BB is disappeared in sham and PR rats, while there is a significant increase in GST-BB in rats chronically treated with alcohol. Plasma ALT levels (57 kDa predicted. M.W) are unchanged in I/R and sham rats. In contrast, ALT in chronic alcohol rat plasma is increased together with a slight shift of immunoreactive bands and appearance of fragments, which may indicate ALT cleavage.

### Example 5

### Proteomic Analysis

Proteomic analysis is applied to the rat I/R liver. Proteins are resolved by biphasic ion-exchange chromatography on a consecutive S- and Q-sepharose columns in tandem with gel electrophoresis (CAX-PAGE). Differential display of proteins are accomplished by Coomassie Blue visible staining, all performed in tandem on the same gels. Proteins with differential expression or modifications are identified using HT analyzer 1D software (Nonlinear Dynamics) and extracted for in-gel trypsin digestion. Digests are analyzed using nanospray liquid chromatography online with tandem mass spectrometry (nano-LC/MSMS). Resulting tandem mass spectra are correlated with tryptic peptide sequences extracted from a non-redundant mammalian protein database utilizing the Sequest algorithm. Several possible homologous proteins are usually generated using this approach. The molecular mass of protein band on SDS-PAGE gel is compared with the predicted molecular masses for sequenced proteins found in databases.

TABLE 1
Biomarkers of liver ischemia/reperfusion injury.

| | Molecular | Cleavage fragments | Change rate in I/R | Detection |
|---|---|---|---|---|
| Liver Biomarker | mass | in I/R liver | liver vs sham | method |
| Argininosuccinate | 46 kDa | 34 kDa, 31 kDa and | 31 and 34 kDa | HTS-WB, |
| synthetase (AS) | | 24 kDa breakdown | expressed in I/R | Western blot |
| | | products by caspase-3 | livers; 24 kDa is | |
| | | | increased >> 10-fold | |
| Squalene synthase | 48 kDa | 36 kDa, cleaved via | Appeared as 36 kDa | HTS-WB |
| (SQS) | | unknown mechanism | in I/R liver | |
| Liver glycogen | 97 kDa | Cleaved; breakdown | 97 kDa band | LC/MS |
| phosphorylase | | products are not | substantially reduced | followed by |
| (GP) | | determined | in I/R liver | sequencing |
| Sulfotransferase | 31 kDa | Cleaved; fragments | 31 kDa nearly | LC/MS |
| (ST) | | are not determined | disappeared in I/R | followed by |
| | | | liver | sequencing |

Samples are also run as tandem for sham-operated liver samples (C) and I/R samples (T). Proteins with differential expression are identified using HT analyzer 1D software (Nonlinear Dynamics) and squared. Protein bands are excised from gel (C band and T hand if present), and digested with trypsin. Digests are analyzed using nano-spray liquid chromatography online with tandem mass spectrometry (nano-LC/MSMS). Resulting tandem mass spectra are correlated with tryptic peptide sequences extracted from a non-redundant mammalian protein database utilizing the Sequest algorithm. Peptide matches only of high spectral correlation are collected by use of DTASelect software data filtering, and IR vs sham liver proteomes are compared using Contrast software. Identification and analysis of most relevant hepatic proteins performed so far is presented below. These proteins are either decreased significantly (e.g. T7A vs. C7A) or disappeared completely (e.g. T4 vs. C4) in I/R liver tissue compared to sham-operated livers.

### Example 6

### Liver Transplant Patients

FIG. 29 shows ASS serum values from six liver transplant patients. Serum samples are collected from liver transplant patients (n=6) before the transplant occurred (baseline), while the liver is removed (ahepatic) and at various time points after the new liver is inserted into the patients (1 or 3 min, 30 min, 45 min, 60 min, 120 min). Serum ASS levels are measured by ASS specific SW ELISA(in ng/ml). Values of 150 ng/ml exceeded the sensitivity of the assay.

TABLE 2
Hepatic proteins identified by nano-spray liquid chromatography with tandem mass
spectrometry (nano-LC/MSMS), which are decreased or disappeared in I/R liver
tissue vs sham operated liver after differential display on SDS-PAGE.

| | | | Predicted | Observed | | | T | |
|---|---|---|---|---|---|---|---|---|
| Band | GI | Protein Name | Mass | Mass | C pep | C % | pep | T % |
| 4 | Gi: 6978809 | enolase 1, | 47.5 | 47 | 4 | 11 | | |
| | | alpha. [Rattus | | | | | | |
| | | norvegicus] | | | | | | |
| 6A | Gi: 1560087 | liver glycogen | 97.9 | 99 | 2 | 2.1 | | |
| | | phosphorylase | | | | | | |
| | | [Rattus | | | | | | |
| | | norvegicus]. | | | | | | |
| 7A | Gi: 11560087 | liver glycogen | 97.9 | 99 | 8 | 17.5 | 2 | 4.8 |
| | | phosphorylase | | | | | | |
| | | [Rattus | | | | | | |
| | | norvegicus]. | | | | | | |
| 10 | Gi: 6981594 | Estrogen | 35.4 | 35 | 2 | 7.1 | | |
| | | Sulfotransferase | | | | | | |
| | | [Rattus | | | | | | |
| | | norvegicus] | | | | | | |
| 12 | Gi: 8393186 | carbamoyl- | 164.6 | 120 | 2 | 1.5 | | |
| | | phosphate | | | | | | |
| | | synthetase 1; | | | | | | |
| | | [;Rattus | | | | | | |
| | | norvegicus] | | | | | | |
| 13 | Gi: 8393186 | carbamoyl- | 164.6 | 120 | 3 | 2.3 | | |
| | | phosphate | | | | | | |
| | | synthetase 1; | | | | | | |
| | | [Rattus | | | | | | |
| | | norvegicus] | | | | | | |
| 14A | Gi: 8392839 | ATP citrate | 121.5 | 120 | 2 | 2.8 | | |
| | | lyase [Rattus | | | | | | |
| | | norvegicus] | | | | | | |
| | Gi: 8393186 | carbamoyl- | 164.6 | 120 | 5 | 4.1 | | |
| | | phosphate | | | | | | |
| | | synthetase 1; | | | | | | |
| | | [Rattus | | | | | | |
| | | norvegicus] | | | | | | |
| 14C | Gi: 8393322 | glucose | 51 | 56 | 6 | 13.1 | | |
| | | regulated | | | | | | |
| | | protein, 58 kDa | | | | | | |
| | | [Rattus | | | | | | |
| | | norvegicus] | | | | | | |

### Example 7

### ASS and ALT as Biomarkers of Chlorinated Hydrocarbon Liver Injury

Rats (n=6) are injected with 0.5 ml/kg of carbon tetrachloride (CCL₄) in vegetable oil. Plasma arginosuccinate synthetase (ASS) and alanine transaminase (ALT) accumulation is measured 1 hr and 24 hr after injection. ASS increased over 15-fold compared to controls after 1 hr and over 50-fold compared to controls after 24 hr (p<0.0001), as shown in FIG. 30A. ALT did not increase significantly after 1 hr but showed a significant increase (p=0.041) 24 hr after injection as shown in FIG. 30B.

### Example 8

### ASS, CPS-1 and SULTA1 as Biomarkers of Ecstasy Liver Injury

Serum arginosuccinate synthetase (ASS), carbamoylphosphate synthase-1 (CPS-1) and sulfur transferase isoform A1 (SULT2A1) levels are measured after repeated injections of 10 mg/kg of methylenedioxyacetamine (MDMA) at 1.5 hr intervals. A significant increase in ASS compared to control serum levels in rats is observed after i.p. injection of 20 mg/kg (p<0.01), after a total of 40 mg/kg (p<0.001), and in SULT2A1 after a total of 20 mg/kg (p<0.001) and 40 mg/kg (p<0.0001), FIG. 31A and FIG. 31B. No increase is observed in alanine transferase (ALT). Rats are sacrificed 24 hr after the last ecstasy injection and CPS-1 is detected by PAGE on 4-12% gels (4 injections of MDMA (10 mg/kg administered 1.5 hr apart). FIG. 32 shows no detectable MDMA in the saline control.

### Example 9

### Biomarkers of Bacterial Endotoxin Liver Injury

Rats are injected with 100 ug/kg of bacterial endotoxin (E-LPS). Serum arginosuccinate synthetase (ASS) significantly increased after 1 hr and remained increased compared to controls at least up to 24 hr after injection, see FIG. 33A. There is no significant increase in ALT or aspartate transaminase (AST) even 24 hr post injection, see FIG. 33B and FIG. 33C.

When the rats are injected with E-LPS and D-galactosamine, 10 ug/kg and 500 mg/kg respectively, ASS levels increased dramatically by 24 hr, showing an increased serum level of >3.7 fold after 1 hr, >200 fold after 3 hr and >1000 fold after 24 hr. ALT levels also increased, peaking at >29-fold at 24 hr. Both ASS recovered to baseline or control levels after 24 hr while ALT decreased to about 4-fold over controls after 72 hr. The levels of ASS and ALT up to 72 hr are shown in FIG. 34A and FIG. 34B.

### Example 10

### Biomarker Assay Development

Anti-biomarker specific rabbit polyclonal antibody and monoclonal antibodies are produced in the laboratory. To determine reactivity specificity of the antibodies to detect a target biomarker a known quantity of isolated or partially isolated biomarker is analyzed or a tissue panel is probed by western blot. An indirect ELISA is used with the recombinant biomarker protein attached to the ELISA plate to determine optimal concentration of the antibodies used in the assay. Microplate wells are coated with rabbit polyclonal anti-human biomarker antibody. After determining the concentration of rabbit anti-human biomarker antibody for a maximum signal, the lower detection limit of the indirect ELISA for each antibody is determined. An appropriate diluted sample is incubated with a rabbit polyclonal antihuman biomarker antibody for 2 hours and then washed. Biotin labeled monoclonal anti-human biomarker antibody is then added and incubated with captured biomarker. After thorough wash, streptavidin horseradish peroxidase conjugate is added. After 1 hour incubation and the last washing step, the remaining conjugate is allowed to react with substrate of hydrogen peroxide tetramethyl benzadine. The reaction is stopped by addition of the acidic solution and absorbance of the resulting yellow reaction product is measured at 450 nanometers. The absorbance is proportional to the concentration of the biomarker. A standard curve is constructed by plotting absorbance values as a function of biomarker concentration using calibrator samples and concentrations of unknown samples are determined using the standard curve.

### Example 11

### Liver Patient Samples

Subjects with suspected liver injury are enrolled at several investigational sites globally. All Subjects receive standard of care treatment when presenting to the investigational site. Biological samples of blood, urine, saliva and CSF are collected from the subjects at specified timepoints. Inclusion criteria for the Subjects include 1) The Subject is at least 18 years of age at screening (has had their 18th birthday) and no more than 80 years of age (did not have their 81st birthday); 2) the Subjects primary diagnosis is a form of liver injury from traumatic liver injury, transplantation injury, toxic liver injury, ischemic liver injury, radiation exposure injury, mechanical liver injury, sepsis, and injury due to exposure to hepatoxic compounds; 3) the biological samples of blood urine and saliva are able to be collected within four (4) hours after injury; 4) proper informed consent from patient or guardian. Follow up samples are taken at several timepoints to monitor injury.

### Example 12

### Normal Patient Samples

Normal Subjects without any known or suspected TBI, liver damage, stroke or other conditions which may alter protein biomarker levels are enrolled at several investigational sites globally. All Subjects receive standard screening to ensure that no medications or ailments are experienced by the patients prior to enrollment into the study. Biological samples of blood, urine, saliva and CSF are collected from the subjects upon enrollment. Inclusion criteria for the Subjects include 1) The Subject is at least 18 years of age at screening (has had their 18th birthday) and no more than 80 years of age (did not have their 81st birthday); 2) the Subject is screened and found to not be taking medications or suffering from any neurological injury, neurological disorder, neurotoxicity, or liver injury; 3) proper informed consent from patient or guardian

### Example 13

### Analysis of Liver Damage Markers

Accumulation of novel markers indicating neurotoxic insult such as Argininosuccinate synthetase (ASS), argininosuccinate lyase (ASL), sulfuration (estrogen sulfotransferase (EST), squalene synthase (SQS), liver glycogen phosphorylase (GP), carbamoyl-phosphate synthetase (CPS-I), α-enolase 1, glucose-regulated protein (GRP) and spectrin breakdown products and combinations thereof, are analyzed in the biological samples taken after TBI using the inventive in vitro diagnostic devices. Normal patient samples are also analyzed for the same biomarkers, and a normal metric is calculated to indicate a non-injury state. The metric is then incorporated into the in vitro diagnostic device either through a computer algorithm, or in the event of a calorimetric indication, the dyes are activated indicating injury when the level of the measured biomarker is higher than what is determined in the normal metric.

Prior to analysis, an assay is developed using a detection and capture antibody, each antibody being specific to the biomarker intended to be measured. For example, for ASS a monoclonal/monoclonal pair (capture/detection) is used to detect the level of biomarkers. Notwithstanding, similar results are achieved through the use of a monoclonal/polyclonal pair, a polyclonal monoclonal pair, and a polyclonal/polyclonal pair. The assay is optimized and tested using a calibrator and spiked serum to ensure that assay can measure known positive and known negative controls and detect the levels of known proteins within 1 picogram/mL detection sensitivity. The assay is incorporated into an in vitro diagnostic device using a cartridge or other disposable, whereby the cartridge contains the assay and a biological sample collection chamber for receiving the biological sample. The present invention further incorporates by reference the antibody and detection methods for the claimed biomarkers being used in the device for the specific indication disclosed therein presented in US 2013/0029362 A1. The in vitro diagnostic devices used in this example have incorporated assays contained therein, which assays may be substituted herein using the methods therein contained.

### Other Embodiments

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. An in vitro diagnostic device for detecting liver damage in a subject, the device comprising:
a sample chamber for holding a first biological sample collected from the subject;
an assay module in fluid communication with said sample chamber, said assay module containing an agent for detecting one or more biomarkers of a neural injury or neuronal disorder selected from the group consisting of: Argininosuccinate synthetase (ASS), argininosuccinate lyase (ASL), sulfuration (estrogen sulfotransferase (EST), squalene synthase (SQS), liver glycogen phosphorylase (GP), carbamoyl-phosphate synthetase (CPS-1), α-enolase 1, SULT2A1, glucose-regulated protein (GRP), wherein said assay module analyzes the first biological sample to detect the amount of the one or more biomarker present in said sample;
a data processing module determines the measured amount of the respective biomarker present in the sample, an indication of the presence or absence of liver injuries, and/or an indication of the severity of liver injuries
a user interface, wherein said user interface relates the amount of the one or more biomarker measured in the assay module to detecting liver damage in the subject or the severity of lover damage in the subject.

2. The device of claim 1, wherein the liver injury is one of: traumatic liver injury, transplantation injury, toxic liver injury, ischemic liver injury, radiation exposure injury, mechanical liver injury, sepsis, and injury due to exposure to hepatoxic compounds.

3. The device of claim 1 wherein said assay module further comprises at least one additional agent selective to measure for at least one additional biomarker selected from the group consisting of: Argininosuccinate synthetase (ASS), argininosuccinate lyase (ASL), sulfuration (estrogen sulfotransferase (EST), squalene synthase (SQS), liver glycogen phosphorylase (GP), carbamoyl-phosphate synthetase (CPS-1), α-enolase 1, SULT2A1, glucose-regulated protein (GRP).

4. The device of claim 1 wherein the first biological sample is selected from the group consisting of blood, blood plasma, serum, sweat, saliva, cerebrospinal fluid (CSF) and urine.

5. The device of claim 1 wherein said assay further comprises a dye providing a colorimetric change in response to the one or more biomarker present in the first biological sample.

6. The device of claim 1 wherein said assay module is an immunoassay.

7. The device of claim 6 wherein the immunoassay is an ELISA.

8. The device of claim 1, wherein said agent is an antibody or a protein.

9. The device of claim 1, further comprising a power supply and a data processing module in operable communication with said power supply and said assay module wherein said data processing module compares an input signal of the measured amount of a biomarker in the sample from the assay module to a preprogrammed threshold level to determine an output.

10. The device of claim 1 wherein an output from the data processing module relates to detecting liver damage in the subject, the output displaying the amount of the one or more biomarker measured in said sample, the output displaying the presence or absence of liver damage, or the output displaying the severity of liver damage.

11. The device of claim 9, further comprising analyzing a second biological sample obtained from the subject, at some time after the first sample is collected, wherein if the device detects a decreased amount of the one or more biomarker in the second sample relative to the first sample a recovery output is provided by the data processing module.

12. The device of claim 9 further comprises a display in electrical communication with said data processing module and displaying the output as at least one of an amount of the one or more biomarker, a comparison between the amount of the one or more biomarker and a control, presence of the liver damage, or severity of the liver damage.

13. The device of claim 9 further comprising a transmitter for communicating the output to a remote location.

14. The device of claim 9 wherein the output is digital.

15. A method for using an in vitro diagnostic device for detecting liver damage in a subject, the method comprising:
calibrating an in vitro diagnostic device incorporating an assay for measuring one or more biomarkers of liver damage in a biological sample, the one or more biomarkers selected from the group consisting of: Argininosuccinate synthetase (ASS), argininosuccinate lyase (ASL), sulfuration (estrogen sulfotransferase (EST), squalene synthase (SQS), liver glycogen phosphorylase (GP), carbamoyl-phosphate synthetase (CPS-1), α-enolase 1, SULT2A1, glucose-regulated protein (GRP);
obtaining a biological sample from a subject;
applying said sample to said in vitro diagnostic device wherein said assay includes reagents to determine the amount of the one or more biomarker present in said sample, wherein said device provides an output which relates the amount of the one or more biomarker detected to liver damage, or lack thereof, in the subject.

16. The method of claim 14 further comprising:
calibrating an in vitro diagnostic device incorporating an assay for additionally measuring at least one additional biomarker selected from the group consisting of: Argininosuccinate synthetase (ASS), argininosuccinate lyase (ASL), sulfuration (estrogen sulfotransferase (EST), squalene synthase (SQS), liver glycogen phosphorylase (GP), carbamoyl-phosphate synthetase (CPS-1), α-enolase 1, SULT2A1, glucose-regulated protein (GRP).
applying said sample to said in vitro diagnostic device wherein said assay includes reagents to determine the amount of the additional biomarker present in said sample, wherein said device provides an output which relates the amount of the additional biomarker detected, alone or in synergistic combination with the one or more biomarker, to liver damage, or lack thereof, in the subject.

17. A method of treating liver damage in a subject:
calibrating an in vitro diagnostic device incorporating an assay for measuring for one or more biomarkers in a biological sample, the one or more biomarkers selected from the group consisting of: (Argininosuccinate synthetase (ASS), argininosuccinate lyase (ASL), sulfuration (estrogen sulfotransferase (EST), squalene synthase (SQS), liver glycogen phosphorylase (GP), carbamoyl-phosphate synthetase (CPS-1), α-enolase 1, SULT2A1, glucose-regulated protein (GRP);
obtaining a biological sample from a subject;
applying said sample to said in vitro diagnostic device wherein said assay includes reagents to determine the amount of the one or more biomarker present in said sample, wherein said device provides an output which relates the amount of the one or more biomarker detected to liver damage, or lack thereof, in the subject, wherein if said output of said in vitro diagnostic device relates the amount of the one or more biomarker to liver damage, a therapeutic intervention is employed to treat injury and/or inhibit injury progression.

18. A process for electronically diagnosing liver injury or liver damage in a subject, the process comprising:
an input signal from an assay module that has measured an amount of a liver biomarker in a biological sample;
a software package providing instructions to a central processing unit for receiving and processing the input signal, comparing the input signal to a database of liver injury biomarker levels to determine if the amount if the input is greater than or less than the database amount stored on a memory unit of a data processing module, and translating the input data into usable indication of the presence or absence of the neurological condition; and
communicating the usable indication to a graphical user interface to display the indication.

19. The process of claim 18 further comprising a network for communicating the usable indication to a remote display database, or computer terminal.

20. The process of claim 18 further comprising saving the usable indication in machine readable format to the memory unit of the data processing module.

21. The process of claim 18 wherein the comparison step is performed by a CPU receiving instructions from a software application.

22. The process of claim 18 wherein the database amount is a threshold level, predetermined for each biomarker respectively, is based on a known positive level of the biomarker, is a known negative level of the biomarker, or is the amount of the biomarker measured in normal control.

23. The process of claim 18 wherein the liver biomarker is one or more biomarkers selected from the group consisting of: (Argininosuccinate synthetase (ASS), argininosuccinate lyase (ASL), sulfuration (estrogen sulfotransferase (EST), squalene synthase (SQS), liver glycogen phosphorylase (GP), carbamoyl-phosphate synthetase (CPS-1), α-enolase 1, SULT2A1, or glucose-regulated protein (GRP).

24. The process of claim 18 wherein the usable indication is the measured amount of the biomarker present in the sample, an indication of the presence or absence of liver injuries, the type of liver injury, or the severity of a liver injury.

25. The process of claim 18 wherein the input is two or more inputs received from at least one assay module of two or more biomarkers measured by the assay module.
